(19) United States
(12) Patent Application Publication
(10) Pub. No.: US 2024/0226591 A1
(43) Pub. Date: Jul. 11, 2024

(54) MAJOR DEPRESSIVE DISORDER OR PERSISTENT DEPRESSIVE DISORDER PREVENTION OR TREATMENT WITH LOW INTENSITY AND HIGH FREQUENCY MAGNETIC STIMULATION

(71) Applicant: Actipulse Neuroscience Inc., Cambridge, MA (US)

(72) Inventor: Alain Andre CHÂTILLON, Mexico City (MX)

(21) Appl. No.: 18/561,535

(22) PCT Filed: May 17, 2022
(86) PCT No.: PCT/US2022/029553

§ 371 Date: Nov. 17, 2023

Related U.S. Application Data
(60) Provisional application No. 63/189,443, filed on May 17, 2021.

(51) Int. Cl. A61N 2/00; A61N 2/02

(52) U.S. Cl. CPC A61N 2/006; A61N 2/02

(57) ABSTRACT

The present disclosure provides, in part, methods of treatment for major depressive disorder comprising applying repetitive transcranial magnetic stimulation (rTMS) therapy to a patient in need thereof. The present disclosure also provides devices that generate a low intensity pulsed magnetic field and variable frequencies.

### CROSS-REFERENCE TO RELATED APPLICATIONS

This application claims the benefit of U.S. Provisional Patent Application No. 63/189,443, filed on May 17, 2021, the entire contents of all of which are hereby incorporated by reference in their entirety.

### FIELD

The present disclosure provides, in part, clinical applications of a non-invasive brain stimulation, e.g. repetitive Transcranial Magnetic Stimulation (rTMS), as it pertains to neuromodulation in the treatment of neurologic and psychiatric disorders and diseases.

### BACKGROUND

Major depressive disorder (MDD) is a debilitating disease characterized by at least 5 of the following symptoms: depressed mood, diminishment of interest or pleasure obtained from the activities, changes in appetite or weight, changes in sleep, psychomotor changes, fatigue, feeling of worthlessness, excessive guilt, cognitive impairment and suicidal ideation which last at least 2 weeks (American Psychiatric Association 2013). Globally, depression affects 264 million people, due to its debilitating character, it produces approximately 32846.7 years lived with disability among the population (GBD 2016 Disease and Injury Incidence and Prevalence Collaborators 2017).

Meanwhile, in the US approximately 17.3 million adults have had one major depressive episode, equivalent to 7.1% of adults; being women the most affected by this disorder compared to men, with 8.7% and 5.3% of individuals affected respectively. Due to its debilitating nature, 11 million adults had at least one major depressive episode with severe impairment, equivalent to 4.5% of all U.S. adults and 63.8% of adults with major depressive disorder (“Major Depression”).

Due to the effects seen on quality of life of individuals, investigation has been prioritized to have a better comprehension of this disorder; however, up until now, a single mechanism that could explain all of the facets of this disease has not been found. Nonetheless, it has been recognized that genetic, environmental, endocrinological and neurological factors could be involved in its development. Due to the multiple factors that can elicit the development of MDD, a single treatment option for all patients has not been developed, however management of depression is based mainly in two options: psychotherapy and pharmacotherapy. On the one hand, psychotherapy has shown effects that can be equivalent to pharmacotherapy, however, some individuals find it challenging to pursue, due to accessibility, costs and time consumption. On the other hand, psychopharmacology has proven effective, as antidepressants can induce neuroplasticity and brain modulation. Despite their effectiveness, issues with tolerability of antidepressant side effects have been pointed out, as well as the existence of patients whose symptoms are resistant to treatment, affecting up to 60% of patients (“Major Depression” n.d.; Otte et al. 2016).

Transcranial magnetic stimulation (TMS) is a non-invasive, painless neurostimulation technique with a high safety profile that has been used as a neurologic and psychiatric treatment. However, the use of repetitive TMS as a treatment for neurocognitive diseases and disorders is expensive, requires a clinical setting, is hard to transport, is complex to use, requires external coolers, and may have severe adverse effects such as seizures. These disadvantages are the consequence of current TMS technology, which uses frequencies from 1 to 50 Hz and intense magnetic fields of around 10 million milligauss (i.e., around 1 Tesla).

There is a need for improved treatments for depression, like MDD. There is also a need for devices and methods that employ a non-invasive brain stimulation, e.g. rTMS, without using large magnetic fields, and without focusing the magnetic waves at a certain point in the brain. There is a further need for a user-friendly device that is portable, appropriate for home use, and is safe to use without the need of a medical professional to apply it.

### SUMMARY

Accordingly, the present disclosure provides low intensity and high frequency non-invasive brain stimulation, e.g. rTMS, methods to safely and effectively treat or prevent major depressive disorder in a convenient manner, directly from home and without clinical supervision.

In one aspect, the present disclosure provides a non-invasive brain stimulation, e.g. rTMS, method for treating or preventing major depressive disorder, comprising repetitively applying a magnetic pulse to the scalp of a patient in need thereof thereby stimulating neurons in the brain of the patient, wherein the magnetic pulse is applied (a) repetitively over the patient's brain (e.g., without limitation, the patient's left prefrontal dorsolateral cortex); and (b) at a frequency of about 100 to about 1000 Hz and an intensity of about 5,000 to about 15,000 milligauss (about 0.0005 Tesla to about 0.0015 Tesla).

In various embodiments, the present disclosure contemplates applying a magnetic pulse using rTMS to a patient afflicted with major depressive disorder or persistent depressive disorder. In various embodiments, the patient with major depressive disorder presents as having at least 5 of the following symptoms: depressed mood most of the time, loss of interest or pleasure obtained from almost all of their activities, changes in appetite that can cause weight changes without planning on it, changes in sleep (insomnia or hypersomnia), psychomotor changes every day (agitation or retardation), fatigue nearly every day, feelings of worthlessness or excessive guilt, diminished cognitive abilities, recurrent thoughts of dead including suicidal ideation; this symptoms should be present for, at least, a 2-week period and cause significant distress or impairment in different areas of functioning. While, persistent depressive disorder—previously known as dysthymia—is characterized as a chronic depressive disorder in which the patient presents depressive mood most of the time for at least 2 years and 2 or more of the following symptoms: changes in appetite, insomnia or hypersomnia, fatigue, low self-esteem, cognitive impairments, feelings of hopelessness; these symptoms should cause significant distress or impairment in different areas of functioning and the patient has never been without symptoms for more than 2 months at a time.

In various embodiments, treating or preventing major depressive disorder comprises diminishing depressive symptoms (e.g. anhedonia, apathy, insomnia, guilt, hopelessness), decreasing anxiety symptoms and/or increasing quality of life in the patient in need thereof.

In various embodiments, treating or preventing major depressive disorder comprises slowing memory loss or retaining or increasing memory capacity, memory function, or cognitive function in the patient in need thereof. In some embodiments, a method of slowing memory loss or retaining or increasing memory capacity, memory function, or cognitive function is provided, comprising administering rTMS described herein to a patient in need thereof.

In some embodiments, depressive symptoms, anxiety symptoms and quality of life can be assessed by using one or more of the Beck Depression Inventory, Hamilton Depression Rating Scale, Montgomery-Asberg Depression Rating Scale, Center for Epidemiologic Studies Depression Scale, Beck Hopelessness Scale, Geriatric Depression Scale, Cornell Dysthymia Rating Scale, Quick Inventory of Depressive Symptomatology, Reminiscence Functions Scale, EQ-5D, Goldberg Anxiety and Depression Scale, Beck Anxiety Inventory, Hamilton Anxiety Rating Scale, Generalized Anxiety Disorder 7-item, Health Anxiety Inventory, The State-Trait Anxiety Inventory, Geriatric Anxiety Inventory, Taylor Manifest Anxiety Scale, Hospital Anxiety and Depression Scale, Anxiety Sensitivity Index, Athens Insomnia Scale, Insomnia Severity Index, Pittsburgh Sleep Quality Index, Sleep Quality Scale, Daytime Insomnia Symptom Scale, Insomnia Symptom Questionnaire, Pittsburgh Insomnia Rating Scale, Global Sleep Assessment Questionnaire, PROMIS Sleep Disturbance, Brief Fatigue Inventory, Epworth Sleepiness Scale, Functional Outcomes of Sleep Questionnaire, Mini Sleep Questionnaire, Bergen Insomnia Scale, Patient Health Questionnaire, Short Form Health Survey, Quality of Life Scale, Social Adjustment Scale-Self Report, McGill Quality of Life Questionnaire, Health-related quality of life, World Health Organization Quality of Life Instrument, Global Quality of Life Scale, Social Functioning Questionnaire, Life Satisfaction Index, Quality Of Life In Depression Scale, Assessment of Quality of Life, Control, Autonomy, Self-Realization and Pleasure (CASP-19), and Patient-Reported Outcomes Measurement Information System, amongst others. In embodiments, the present methods provide improvement in a patient or population of patients, as assessed by the aforementioned scales of this paragraph.

In some embodiments, memory capacity, memory function, cognitive function, or memory loss is assessed using one or more of the Montreal Cognitive Assessment (MoCA), Minimental state examination (MMSE), Verbal fluidity test, Frontal Assessment Battery (FAB), Geriatric Depression Scale (GDS-15), Clinical dementia rating (CDR), EuroQoL-5D, Daily Life Activities of Katz (ABVD), Lawton Daily Life Instrumental Activities Scale (AIVD), and Bayer Scale of Activities of Daily Living (B-ADL), Clinical Dementia Rating-Sum of Boxes (CDR-SB), and Repeatable Battery for the Assessment of Neuropsychological Status (RBANS). In embodiments, the present methods provide improvement in a patient or population of patients, as assessed by the aforementioned scales of this paragraph.

### DESCRIPTION OF THE FIGURES

FIG. 1A-C depicts the enclosure of a non-limiting example of the rTMS device. FIG. 1A shows the front view of the enclosure. FIG. 1B shows the back view of the enclosure. FIG. 1C depicts the top view of the enclosure.

FIG. 2 depicts a headband that fits a non-limiting example of the rTMS device on a head.

FIG. 3 depicts a non-limiting example of the proposed use of the rTMS device. It shows the first part described in FIG. 1A-C and headband described in FIG. 2.

FIG. 4 depicts the changes in several MDD and PDD related scores before and after 15 sessions of treatment

FIG. 5 depicts a non-limiting example of a pattern of stimulation provided by the rTMS device.

### DETAILED DESCRIPTION

In various aspects, the present disclosure provides, in part, methods for the treatment, prevention, or improvement of depressive symptoms, anxiety symptoms, quality of life and cognitive performance in patients with MDD, or persistent depressive disorder (PDD) using high frequency, low intensity transcranial magnetic stimulation and/or non-invasive brain stimulation using high frequency, low intensity magnetic fields.

Repetitive Transcranial Magnetic Stimulation (rTMS)

In embodiments, the present disclosure provides methods for using non-invasive brain stimulation.

In various embodiments, the present disclosure provides for using transcranial magnetic stimulation (TMS) in a non-invasive and non-painful method to stimulate the cerebral cortex. In embodiments, the present disclosure contemplates a device that generates low-intensity pulsed magnetic fields and high frequencies. In some embodiments, rTMS activates or inhibits cortical activation by generating a magnetic field that, by Faraday's principle, generates an electrical field inside the brain. In some embodiments, the magnetic field is produced when an electric current passes through a coil. Further, in some embodiments, in order to generate an electric field inside the brain, the magnetic field starts and finishes rapidly. Without wishing to be bound by theory, if the magnetic field intensity stays constant too long, no electrical changes occur inside the brain. In addition, it is contemplated that when making use of magnetic fields of lower magnitude, neither a voltage transformer nor high currents are necessary. Indeed, the present disclosure contemplates a device that decreases potential adverse effects, facilitates its transport, and does not require external coolers for the operation.

In various embodiments, the present disclosure provides for using transcranial magnetic stimulation (TMS), wherein the magnetic pulse is applied at a frequency of about 100 to about 1000 Hz, about 100 to about 900 Hz, about 100 to about 800 Hz, about 100 to about 700 Hz, about 100 to about 600 Hz, about 100 to about 500 Hz, about 100 to about 400 Hz, about 100 to about 300 Hz, about 100 to about 200 Hz, about 200 to about 1000 Hz, about 300 to about 1000 Hz, about 400 to about 1000 Hz, about 500 to about 1000 Hz, about 600 to about 1000 Hz, about 700 to about 1000 Hz, about 800 to about 1000 Hz, or about 900 to about 1000 Hz. In specific embodiments, the magnetic pulse is applied at a frequency of about 100 Hz, about 200 Hz, about 300 Hz, about 400 Hz, about 500 Hz, about 600 Hz, about 700 Hz, about 800 Hz, about 900 Hz, or about 1000 Hz.

In embodiments, the present disclosure provides for using transcranial magnetic stimulation (TMS), wherein the magnetic pulse is applied at an intensity of about 1,000 to about 20,000 milligauss, or about 5,000 to about 15,000 milligauss, or about 7,000 to about 12,000 milligauss. In embodiments, the present disclosure provides for using transcranial magnetic stimulation (TMS), wherein the magnetic pulse is applied at an intensity of about 1,000 to about 20,000 milligauss, or about 1,000 to about 15,000 milligauss, or about 1,000 to about 10,000 milligauss, or about 1,000 to about 5,000 milligauss, or about 5,000 to about 15,000 milligauss, or about 10,000 to about 15,000 milligauss. In embodiments, the magnetic pulse is applied at an intensity of about 1,000 milligauss, about 2,000 milligauss, about 3,000 milligauss, about 4,000 milligauss, about 5,000 milligauss, about 6,000 milligauss, about 7,000 milligauss, about 8,000 milligauss, about 9,000 milligauss, about 10,000 milligauss, about 11,000 milligauss, about 12,000 milligauss, about 13,000 milligauss, about 14,000 milligauss, about 15,000 milligauss, about 16,000 milligauss, about 17,000 milligauss, about 18,000 milligauss, about 19,000 milligauss, or about 20,000 milligauss.

In embodiments, the present disclosure provides for using transcranial magnetic stimulation (TMS), wherein the magnetic pulse is applied at an intensity of about 0.0001 to about 0.002 Tesla, or about 0.0005 to about 0.0015 Tesla, or about 0.0007 to about 0.0012 Tesla. In embodiments, the magnetic pulse is applied at an intensity of about 0.0001 Tesla, about 0.0002 Tesla, about 0.0003 Tesla, about 0.0004 Tesla, about 0.0005 Tesla, about 0.0006 Tesla, about 0.0007 Tesla, about 0.0008 Tesla, about 0.0009 Tesla, about 0.001 Tesla, about 0.0011 Tesla, about 0.0012 Tesla, about 0.0013 Tesla, about 0.0014 Tesla, about 0.0015 Tesla, about 0.0016 Tesla, about 0.0017 Tesla, about 0.0018 Tesla, about 0.0019 Tesla, or about 0.002 Tesla.

In embodiments, the present disclosure provides for using transcranial magnetic stimulation (TMS), wherein the magnetic pulse generates an electric field of about 0.1 to about 10 V/m², about 0.5 to about 5 V/m², or about 0.5 to about 1.5 V/m². In embodiments, the magnetic pulse generates an electric field of about 1 V/m⁷.

In embodiments, the rTMS treatment method is undertaken once, or twice, or thrice, or four times daily. In particular embodiments, the method is undertaken once, twice, or thrice daily. In embodiments, the rTMS treatment method is undertaken for greater than about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments, the method is undertaken for about 15 to about 60 minutes. In particular embodiments, the method is undertaken for about 30 minutes. In various embodiments, the rTMS treatment method comprises magnetic pulses that are applied about 200 to about 1000 times, or about 200 to about 900 times, or about 200 to about 800 times, or about 200 to about 700 times, or about 200 to about 600 times, or about 200 to about 500 times, or about 200 to about 400 times, or about 200 to about 300 times, or about 300 to about 1000 times, or about 300 to about 900 times, or about 300 to about 800 times, or about 300 to about 700 times, or about 300 to about 600 times, or about 300 to about 500 times, or about 300 to about 400 times. In various embodiments, the rTMS treatment method comprises magnetic pulses that are applied about 300 to about 400 times. In certain embodiments, the pulses are applied about 300 times, about 310 times, about 320 times, about 330 times, about 340 times, about 350 times, about 360 times, about 370 times, about 380 times, about 390 times, or about 400 times. In various embodiments, the pulses last for about two seconds, about three seconds, about four seconds, or about five seconds. In certain embodiments, the pulses last for about three seconds, followed by one second without pulsing. In embodiments, a pulse is applied for a period of three seconds, followed by a one second pause in which no pulse is applied.

In various embodiments, the rTMS treatment method contemplated by the present disclosure is applied chronically. For example, in some embodiments, the treatment is applied for greater than about 1 month, about 2 months, about 3 months, about 4 months, about 5 months, about 6 months, about 7 months, about 8 months, about 9 months, about 10 months, about 11 months, or about 1 year, or about 2 years, or about 3 years, or about 5 years, or about 10 years. In some embodiments, the treatment is applied for the life of the patient. In some embodiments, the treatment is self-applied.

In embodiments, the rTMS treatment method contemplated by the present disclosure is applied to the patient until symptoms, such as those described herein, improve or diminish. For example, the patient may undergo the treatment and experience a recovery of depressive symptoms and hope to terminate the treatment. The patient may re-start the treatment if depressive symptoms reemerge. Alternatively, the patient may undergo the treatment and experience a slowing or halting of depressive symptoms, but not an improvement. In such a situation, in embodiments, the patient continues treatment chronically to mitigate or prevent further depressive symptoms.

rTMS Device

In some embodiments, the present disclosure provides devices and equipment for TMS, comprising an electromagnetic field generator and a coil that emits said electromagnetic field. In further embodiments, the magnetic field is produced when an electric current passes through a coil. In some embodiments, pulses of TMS are applied repeatedly with a frequency and a determined magnitude over a cortical area in order to modulate the cerebral cortex activity in long term. In such embodiments, the method is rTMS.

It will be appreciated that the methods of the present disclosure are applied via a device capable of administering the transcranial magnetic stimulation. Indeed, in some embodiments, the TMS is applied using a device that is suitable for conducting electric current through a coil, thus generating a magnetic field. In embodiments, the device is suitable for home use and is optionally portable. In some embodiments, the device comprises a touch screen and optionally includes “digital health” applications and tele-health solutions. In some embodiments, the device is connected via Bluetooth to a smartphone.

In a non-limiting example, the present disclosure contemplates a device that generates a low intensity pulsed magnetic field and variable frequencies. In some embodiments, the device is a transcranial magnetic stimulation system able to generate pulsed magnetic fields in a frequency range from 100 to 1000 Hz. In further embodiments, the device is a transcranial magnetic stimulation system that generates a magnetic field with an intensity from 1,000 to 15,000 milligauss.

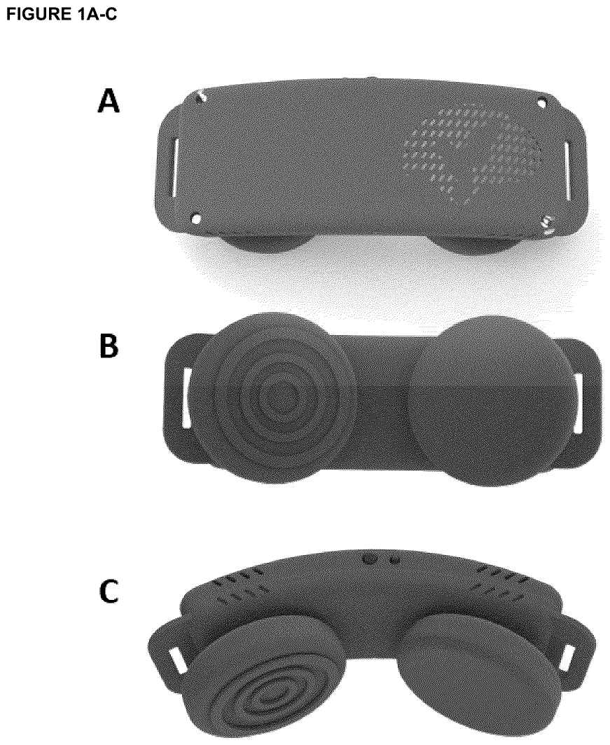

In an additional non-limiting embodiment, the TMS-generating device comprises three parts. In an embodiment, the first part of the device comprises an electronic unit and a coil that are located inside a plastic enclosure as depicted in FIG. 1A. In embodiments, the enclosure has two main faces which are described below. In certain embodiments, the front face has ventilation holes and depicts the correct position of the device. In embodiments, the back face comprises two circular structures that contact the prefrontal part of the skull. In embodiments, in the left circular structure, the coil is located inside (FIG. 1B), while the right circular structure does not have a coil. In embodiments, the electronics inside the plastic enclosure box include a PCB (Print Card Board), in which all the electronic components responsible to provide the electromagnetic waves to the coil by the connector are soldered, a coil and a battery.

In embodiments, the electronic circuit is composed of four parts: (1) a pulse generator from 100 to 1000 Hz, (2) an interruption generator, (3) control of light-emitting diodes (LEDs), (4) and a battery charging circuit. The electric current passes 100 to 1000 times per second through the coil and the flux is interrupted every 3 seconds for 1 second. In embodiments, the circuit is able to generate a pulsed magnetic field with an approximate intensity of 5,000 to 20,000 milligauss. In embodiments, the board has transistors, resistors, heat sinks, electrolytic capacitors, and diodes in order generate the pulse to the given frequency.

In embodiments, a light-emitting diode (LED) is turned on to show the device is connected to the power adapter (5v/2-3 Amp external power supply connected to a 110/220V socket). In an embodiment, a light-emitting diode (LEDs) turn on to show that the device is working. In embodiments, a switch ON/OFF allows the user to start a treatment session.

In embodiments, the coil that transmits the magnetic pulse is located inside the plastic enclosure and over the left prefrontal dorsolateral cortex. In some embodiments, the device comprises a coil of 3-5 centimeters in diameter built from 50-100 turns of copper wire 99.5% pure, gauge 26 AWG. In further embodiments, a double coverage of insulating material covers the coil winding.

In embodiments, a battery or batteries are located inside the plastic enclosure and are used as a power source. The power source supplies the electronics and coil with the necessary current to operate the device and generate the magnetic pulses intended to be applied over the skull.

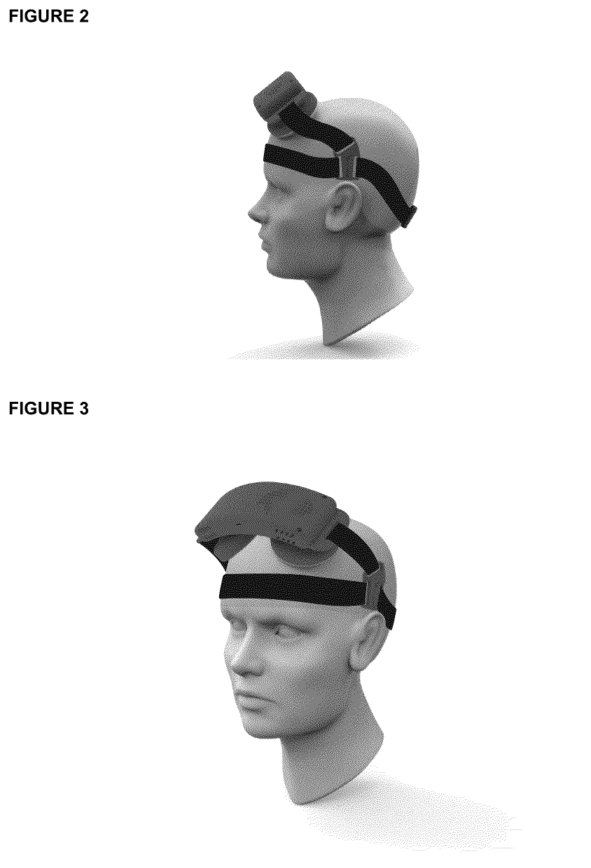

In embodiments, the second part which comprises the device is a headband that allows to place the plastic enclosure in the correct position over the skull, as depicted in FIG. 2. In a non-limiting embodiment, this headband can be fabricated from plastic, steel, aluminum, elastic fabric or other materials. In other embodiments, the headband can have fabric in the side that fits the skull in order to comfortably fit the subject's head.

Finally, in a non-limiting embodiment, the third part of the device comprises a power adapter. In certain embodiments, the power adapter provides an alternate current to direct current transformer, and its input is from 120 to 240 Volts with a frequency of 60 Hz, and its output is from 5V and 1-3 Amperes.

### Methods of Treatment

In certain aspects, the present disclosure contemplates methods for slowing or preventing a conversion of MDD to PDD. In another aspect, the present disclosure contemplates methods for treating or preventing MDD using non-invasive brain stimulation, e.g. rTMS. In various embodiments, the method of rTMS comprises repetitively applying a magnetic pulse to the scalp of a patient in need thereof, thereby stimulating neurons in the brain of the patient, wherein the magnetic pulse is applied (a) repetitively over the patient's brain (e.g., without limitation, the patient's left prefrontal dorsolateral cortex); and (b) at a frequency of about 100 to about 1000 Hz and an intensity of about 1,000 to about 15,000 milligauss. In various embodiments, methods of the present disclosure are suitable for home use and can be performed without the need for a medical professional.

In various embodiments, rTMS methods of the present disclosure stimulate neurons around about 1 to about 4 cm from the skull, about 2 to about 4 cm from the skull, about 1 to about 3 cm from the skull, or about 2 to about 3 cm from the skull. In some embodiments, the method stimulates neurons throughout the patient's brain. In further embodiments, the method substantially stimulates neurons in the left hemisphere of the patient's brain. In still further embodiments, the method substantially stimulates neurons in the frontal lobe of the patient's brain. In embodiments, the method substantially stimulates neurons in the cerebral cortex of the patient. In embodiments, the method stimulates neurons outside of the patient's left prefrontal dorsolateral cortex.

In various embodiments, the present disclosure provides for rTMS treatment methods that prevent or delay the progression of MDD to PDD. In some embodiments, the treatment method improves and/or prevents depressive symptoms and traits of the patient. Further embodiments of the present disclosure include methods for treating psychiatric symptoms linked to MDD and PDD, including, but not limited to, eating disorders, anxiety, and sleep troubles.

In various embodiments, the present treating or preventing includes treatment-of refractory depression, optionally which comprises reduction in length of a depressive episode. In various embodiments, the preventing or treating comprises recovery or improving of an anti-depressive effect of the patient's pre-existent anti-depression treatment regimen. In still other various embodiments, the preventing or treating comprises a reduction in the rate of relapse after major depressive episodes. In further various embodiments, the preventing or treating breakthrough depression and/or treatment-refractory depression comprises prevention or reversal of loss of efficacy of the patient's pre-existent anti comprises reduction in an effective dosage of the patient's pre-existent anti-depression treatment, which may, for example, causes one or more of a reduction in side effects and an increase in patient adherence.

### Patient Selection

In various embodiments, the present disclosure provides treatment of a patient that is afflicted with MDD, including MDD with specifiers as with anxious distress, with melancholic features, with atypical features, with mood-congruent psychotic features, with mood-incongruent psychotic features, with catatonia, with peripartum onset, with season pattern or mixed features.

In various embodiments, the present disclosure provides treatment of a patient that is afflicted with PDD, including PDD with specifiers as with anxious distress, with melancholic features, with atypical features, with mood-congruent psychotic features, with mood-incongruent psychotic features, with catatonia, with peripartum onset, with season pattern or mixed features.

In some embodiments, the patient afflicted with MDD or PDD presents as having the diagnostic criteria specified by the 5th edition Diagnostic and Statistical Manual of Mental Disorders. some embodiments, the present disclosure relates to a method of treating MDD or PDD.

In some embodiments, the present disclosure relates to a method of treating depression, including by way of non-limiting example, breakthrough depression and/or treatment-refractory depression.

In further embodiments, the patient presents as having a test score of one or more identified instruments for assessment of MDD symptoms that is considered to indicate the presence of MDD. For example, in embodiments, the patient presents as having a Beck depression inventory score of 10 or more points; or a Hamilton depression rating scale of 17 or more.

The efficacy of treating depression using methods and compositions of the present disclosure may be assessed by various methods. For example, information about grading of clinical effect can be found in Cusin, Yang, Yeung, and Fava, Rating Scales for Depression (Chapter 2) in Handbook of Clinical Rating Scales and Assessment in Psychiatry and Mental Health (Humana, 2010), Baer and Blias, eds., the contents of which are hereby incorporated by reference in their entirety.

In further embodiments, the patient presents as having a test score of one or more identified instruments for assessment of MDD or PDD symptoms that is considered to indicate the presence of pathology, such tests being found in Table 1 herein

TABLE 1
Instruments for assessment of AD symptoms

| Aspects of MDD and PDD | Abbreviation | Full name |
|---|---|---|
| Depressive symptoms | BDI | Beck Depression Inventory |
| | HDRS | Hamilton Depression Rating Scale |
| | MADRS | Montgomery-Asberg Depression Rating Scale |
| | CES-D | Center for Epidemiologic Studies Depression Scale |
| | GDS | Geriatric Depression Scale |
| | QIDS | Quick Inventory of Depressive Symptomatology |
| | RFS | Reminiscence Functions Scale |
| | CDRS | Cornell Dysthymia Rating Scale |
| | BHS | Beck Hopelessness Scale |
| | Zung SDS | Zung Self-Rating Depression Scale |
| | PROMIS-D | PROMIS Depression Subscale |
| Anxiety Symptoms | BAI | Beck Anxiety Inventory |
| | HARS | Hamilton Anxiety Rating Scale |
| | HAI | Health Anxiety Inventory |
| | STAI | State-Trait Anxiety Inventory |
| | GAI | Geriatric Anxiety Inventory |
| | ASI | Anxiety Sensitivity Index |
| | TMAS | Taylor Manifest Anxiety Scale |
| Anxiety and Depressive | GADS | Goldberg Anxiety and Depression Scale |
| Symptoms | | |
| | HADS | Hospital Anxiety and Depression Scale |
| Sleep Symptoms | AIS | Athens Insomnia Scale |
| | ISI | Insomnia Severity Index |
| | PSQI | Pittsburgh Sleep Quality Index |
| | SQS | Sleep Quality Scale |
| | DISS | Daytime Insomnia Symptom Scale |
| | ISQ | Insomnia Symptom Questionnaire |
| | PIRS | Pittsburgh Insomnia Rating Scale |
| | GSAQ | Global Sleep Assessment Questionnaire |
| | PROMIS-SD | PROMIS Sleep Disturbance subscale |
| | BFI | Brief Fatigue Inventory |
| | ESS | Epworth Sleepiness Scale |
| | FOSQ | Functional Outcomes of Sleep Questionnaire |
| | MSQ | Mini Sleep Questionnaire |
| | BIS | Bergen Insomnia Scale |
| Cognitive Impairment | MMSE | Mini-Mental State Examination |
| | MoCA | Montreal Cognitive Assessment |
| | CAMCOG | Cambridge Cognitive Examination |
| | Mini-Cog | Mini-Cog |
| | CDT | Clock Drawing Test |
| | COGNISTAT | Neurobehavioral Cognitive Status Examination |
| | ACE | Addenbrooke's Cognitive Examination |
| | WAIS-R | Wechsler Adult Intelligence Scale-Revised |
| | Qmci | Quick Mild Cognitive Impairment Screen |
| | NTB | Neuropsychological Test Battery |
| | GPCOG | General Practitioner Assessment of Cognition |
| Quality of Life | PHQ-9 | Patient Health Questionnaire |
| | SF-36 | Short Form Health Survey |
| | QOLS | Quality of Life Scale |
| | SAS-SR | Social Adjustment Scale-Self Report |
| | MQOL | McGill Quality of Life Questionnaire |
| | HRQoL | Health-Related Quality of Life |
| | WHOQOL-BREF | World Health Organization Quality of Life Instrument |
| | GQOL | Global Quality of Life Scale |
| | SFQ | Social Functioning Questionnaire |
| | LSI | Life Satisfaction Index |
| | QLDS | Quality of Life in Depression Scale |
| | AQoL | Assessment of Quality of Life |
| | EQ-5D | EQ-5D |
| | CASP-19 | Control, Autonomy, Self-Realization and Pleasure |
| | PROMIS | Patient-Reported Outcomes Measurement Information |
| | | System |

In some embodiments, a patient that has been treated with rTMS described herein shows an improvement (i.e., an increase or a decrease) in the patient's score of one or more instruments described in Table 1 as compared to baseline (e.g., prior to treatment or at an earlier time point during treatment). In some embodiments, the patient's score of one or more instruments described in Table 1 increases or decreases by at least about 5%, or by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%. In some embodiments, a patient that has been treated with rTMS described herein shows a slowing of the rate of increase or decrease in their score of one or more instruments described in Table 1 by at least about 5%, or by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%. In some embodiments, a stable score of one or more instruments described in Table compared to baseline (e.g., prior to treatment or at an earlier time point during treatment) may be indicative of improvement, slowing, delay, or cessation of MDD and PDD, or a lack of appearance of new clinical, functional, or depressive symptoms or impairments, or an overall stabilization of disease activity.

In some embodiments, efficacy of treatment on depression is assessed by the Hamilton Rating Scale for Depression, for example, a 17-item or 28-item Hamilton Rating Scale for Depression (HAM-D-17 or HAM-D-28). In an embodiment, efficacy may be demonstrated by a reduction in HAM-D-17 score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a HAM-D-17 score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a HAM-D-17 score of greater than about 23, or between about 19-23, or between about 14-18, or between about 8-13. In some embodiments. In some embodiments, the HAM-D-17 score effect size between patients not receiving low dose rTMS and patients receiving rTMS is about 30, or about 25, or about 20, or about 15, or about 14, or about 13, or about 12, or about 11, or about 10, or about 9, or about 8, or about 7, or about 6, or about 5. In some embodiments, the HAM-D-17 score effect size between patients not receiving low dose rTMS and patients receiving rTMS is between about 5 to about 15 or about 5 to about 10. In some embodiments, the rTMS causes any of the above reductions in HAM-D-17 score within about 7 days, or about 10 days, or about 14 days from initiation of treatment with rTMS. In some embodiments, any of the above reductions in HAM-D-17 score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year). In an embodiment, efficacy may be demonstrated by a reduction in HAM-D-28 score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a HAM-D-28 score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a HAM-D-28 score of greater than about 23, or between about 19-23, or between about 14-18, or between about 8-13. In some embodiments. In some embodiments, the HAM-D-28 score effect size between patients not receiving rTMS and patients receiving rTMS is about 30, or about 25, or about 20, or about 15, or about 14, or about 13, or about 12, or about 11, or about 10, or about 9, or about 8, or about 7, or about 6, or about 5. In some embodiments, the HAM-D-28 score effect size between patients not receiving rTMS and patients receiving rTMS is between about 5 to about 15 or about 5 to about 10. In some embodiments, the rTMS causes any of the above reductions in HAM-D-28 score within about 7 days, or about 10 days, or about 14 days from initiation of treatment with rTMS. In some embodiments, any of the above reductions in HAM-D-28 score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In some embodiments, efficacy of treatment on depression is assessed by the Montgomery-Asberg Depression Rating Scale, for example, a 10-item or 15-item Hamilton Rating Scale for Depression (MADRS-10 or MADRS-15). In an embodiment, efficacy may be demonstrated by a reduction in MADRS-10 score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a MADRS-10 score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a MADRS-10 score of greater than about 34, or between about 20-34, or between about 7-19. In some embodiments. In some embodiments, the MADRS-10 score effect size between patients not receiving rTMS and patients receiving rTMS is about 40, or about 35, or about 30, or about 25, or about 20, or about 15, or about 14, or about 13, or about 12, or about 11, or about 10, or about 9, or about 8, or about 7, or about 6, or about 5. In some embodiments, the MADRS-10 score effect size between patients not receiving rTMS and patients receiving rTMS is between about 5 to about 15 or about 5 to about 10. In some embodiments, the rTMS causes any of the above reductions in MADRS-10 score within about 7 days, or about 10 days, or about 14 days from initiation of treatment with rTMS. In some embodiments, any of the above reductions in MADRS-10 score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In an embodiment, efficacy may be demonstrated by a reduction in MADRS-15 score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a MADRS-15 score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a MADRS-15 score of greater than about 34, or between about 20-34, or between about 7-19. In some embodiments. In some embodiments, the MADRS-15 score effect size between patients not receiving rTMS and patients receiving rTMS is about 40, or about 35, or about 30, or about 25, or about 20, or about 15, or about 14, or about 13, or about 12, or about 11, or about 10, or about 9, or about 8, or about 7, or about 6, or about 5. In some embodiments, the MADRS-15 score effect size between patients not receiving rTMS and patients receiving rTMS is between about 5 to about 15 or about 5 to about 10. In some embodiments, the rTMS causes any of the above reductions in MADRS-15 score within about 7 days, or about 10 days, or about 14 days from initiation of treatment with rTMS. In some embodiments, any of the above reductions in MADRS-15 score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In some embodiments, efficacy of treatment on depression is assessed by the Clinical Global Impressions-Severity and Improvement (CGI-S and CGI-I) score. In an embodiment, efficacy may be demonstrated by a reduction in CGI-S score to about 4, about 3.5, about 3, about 2.5, about 2, about 1.5, about 1, or about 0.5, which signify clinical response. In an embodiment, efficacy may be demonstrated by a reduction in CGI-I score to about 4, about 3.5, about 3, about 2.5, about 2, about 1.5, about 1, or about 0.5, which signify clinical response and improvement. In some embodiments, the pre-treatment patient has a CGI-S or CGI-I score effect size between patients not receiving rTMS and patients receiving rTMS is about 1.5, or about 1.3, or about 1.0, or about 0.7, or about 0.5. In some embodiments, the rTMS causes any of the above reductions in CGI-S or CGI-I score within about 7 days, or about 10 days, or about 14 days from initiation of treatment with rTMS. In some embodiments, any of the above reductions in CGI-S or CGI-I score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In various embodiments, the EuroQol 5-D (EQ-5D) is a questionnaire composed of three parts that allow exploring the quality of life of patients. The first part allows the respondent to define the state of health through five dimensions: mobility, personal care, daily activities, pain/discomfort, and anxiety/depression. Each dimension has three levels of severity (no problems, some problems or moderate problems and more serious problems). The combination of the values of all dimensions generates a 5-digit code that determines the patient's health profile. The second part is a visual scale graduated from 0 to 100, where the greater the better state of health perceived. The third part collects other data that allow the demographic characterization of the studied group. This scale has shown satisfactory convergent validity compared to other measures of quality of life, as well as adequate discriminant validity. The test-retest reliability is, overall, moderate to good.

In some embodiments, a patient that has been treated with rTMS described herein shows an improvement (i.e., a decrease) in the patient's EQ-5D five-digit score compared to baseline (e.g., prior to treatment or at an earlier time point during treatment). In some embodiments, the patient's EQ-5D five-digit score decreases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of increase in their EQ-5D five-digit score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable EQ-5D five-digit score compared to baseline (e.g., prior to treatment or at an earlier time point during treatment) may be indicative of a slowing, delay, or cessation of the progression of MDD or PDD, or a reduction in clinical or decline.

In some embodiments, the Mini-Mental State Examination (MMSE) provides a 30-point questionnaire that measures cognitive impairment. In embodiments, any MMSE score of 24 or more (out of 30) indicates a normal cognition. Below this, scores indicate severe (≤9 points), moderate (10-18 points) or mild (19-23 points) cognitive impairment. The raw score may also need to be corrected for educational attainment and age.

In some embodiments, a patient that has been treated with rTMS described herein shows an improvement (i.e., an increase) in the patient's MMSE score compared to baseline (e.g., prior to treatment or at an earlier time point during treatment). In some embodiments, the patient's MMSE score increases by at least 5%, or by at least 10%, or by at least 15%, or by at 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their MMSE score by at least 5%, or by at least 10%, or by at least 15%, at least 20%, or by at least 25%, or by at least 30%, or by at least 35%, or by at least 40%, or by at least 45%, or by at least 50%. In some embodiments, a stable MMSE score compared to baseline (e.g., prior to treatment or at an earlier time point during treatment) may be indicative of a slowing, delay, or cessation of the progression of MDD or PDD, or a reduction in clinical decline.

### Additional Therapeutic Agents and Combination Therapy

Administration of rTMS may be combined with additional therapeutic agents. Co-administration of the additional therapeutic agent and the present rTMS may be simultaneous or sequential.

Further, the present methods provide for treatments of patients that are undergoing treatment with one or more In various embodiments, the additional therapeutic agent as described herein.

In one embodiment, the additional therapeutic agent and the rTMS are administered to a subject simultaneously. The term “simultaneously” as used herein, means that the additional therapeutic agent and the rTMS are administered with a time separation of no more than about 60 minutes, such as no more than about 30 minutes, no more than about 20 minutes, no more than about 10 minutes, no more than about 5 minutes, or no more than about 1 minute.

In a further embodiment, the additional therapeutic agent and the rTMS are administered to a subject simultaneously but the release of the additional therapeutic agent from its respective dosage form and the rTMS may occur sequentially.

Co-administration does not require the additional therapeutic agent and the rTMS to be administered simultaneously, if the timing of their administration is such that the pharmacological activities of the additional therapeutic agent and the administration of rTMS overlap in time. For example, the additional therapeutic agent and the rTMS can be administered sequentially. The term “sequentially” as used herein means that the additional therapeutic agent and the rTMS are administered with a time separation of more than about 60 minutes. For example, the time between the sequential administration of the additional therapeutic agent and the administration of rTMS can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent being administered. Either the additional therapeutic agent or the rTMS may be administered first.

In various embodiments, the present disclosure contemplates rTMS methods used in tandem with one or more additional therapeutic agents. In certain embodiments, the rTMS methods of the present disclosure obviate the need for treatment with one or more additional therapeutic agents. In some embodiments, the additional therapeutic agent is selected from neurological drugs, corticosteroids, antibiotics, antiviral agents, serotonine reuptake inhibitors, Serotonin-norepinephrine reuptake inhibitors, tricyclic antidepressants, dopamine agonists, dopamine antagonists, typical antipsychotics, atypical antipsychotics, serotonin agonists, serotonin antagonists, Norepinephrine reuptake inhibitors, Norepinephrine-dopamine reuptake inhibitors, Monoamine oxidase inhibitors, NMDA receptor antagonists, esketamine, benzodiazepine and baclofen. In specific embodiments, the additional therapeutic agent is a treatment for MDD, including, but not limited to, Fluoxetine, sertraline, vortioxetine, escitalopram and amitriptilina. In various embodiments, the rTMS treatment of the present disclosure is substantially free of adverse effects, optionally selected from epileptic seizures, nausea, and headache.

In embodiments, the additional therapeutic agent is a dopamine active anti-depressant agent. In embodiments, the additional therapeutic agent is one or more of bupropion, aripiprazole, and sertraline. In some embodiments, aripiprazole is not an anti-depressant agent per se. In another embodiment, the SNRT is selected from duloxetine, venlafaxine, nefazodone, and milnacipran. In another embodiment, the dopamine active augmenting agent is one or more of an amphetamine salt, pramipexole, and ropinirole. In another embodiment, the SSRI is selected from citalopram, dapoxetine, s-citalopram, fluoxetine, fluvoxamine, indalpine, paroxetine, and zimelidine.

In some embodiments, the dopamine active anti-depressant agent is one or more of bupropion, aripiprazole, brexpiprazole, and sertraline. Bupropion ((+)-2-(tert-Butylamino)-1-(3-chlorophenyl)propan-1-one), without wishing to be bound by theory, may have its primary pharmacological action through norepinephrine-dopamine reuptake inhibition. It binds selectively to the dopamine transporter, but its behavioral effects may be attributed to its inhibition of norepinephrine reuptake. It also may act as a nicotinic acetylcholine receptor antagonist. Aripiprazole (7-{4-[4-(2,3-Dichlorophenyl)piperazin-1-yl]butoxy}-3,4-dihydroquinolin-2(1H)-one) and brexpiprazole (7-{4-[4-(1-benzothiophen-4-yl)piperazin-1-yl]butoxy}quinolin-2(1H)-one)), without wishing to be bound by theory, are partial dopamine agonist of the second generation class of atypical antipsychotics with additional antidepressant properties that is used in the treatment of schizophrenia, bipolar disorder, and clinical depression. It is approved by the U.S. FDA and EMA for various uses. Without wishing to be bound by theory, Aripiprazole is a dopamimetic at low doses; for example, below 10 mg, or below 9 mg, or below 8 mg, or below 7 mg, or below 6 mg, or below 5 mg, or below 4 mg, or below 3 mg, or below 2 mg, or below 1 mg. Sertraline ((1S,4S)-4-(3,4-dichlorophenyl)-N-methyl-1,2,3,4-tetrahydronaphthalen-1-amine) is a compound of various mood disorder mechanisms. Without wishing to be bound by theory, it may be a dopamine active antidepressant; for example, at doses of 150 mg or above (for example, 160, or 170, or 180, or 190, or 200 mg). Without wishing to be bound by theory, it may also be an antidepressant of the selective serotonin reuptake inhibitor (SSRI) class. It is approved by the U.S. FDA. In some embodiments, the present disclosure provides methods and compositions comprising doses of sertraline at doses at which sertraline acts as an inhibitor of dopamine uptake. For example, the present disclosure encompasses doses of sertraline of doses of above 150 mg daily or above 200 mg daily or above 250 mg daily.

In some embodiments, the additional therapeutic agent comprises serotonin-norepinephrine reuptake inhibitors (SNRIs). SNRIs include agents which act upon, and increase, the levels of the neurotransmitters serotonin and norepinephrine, which play an important role in mood. In some embodiments, the SNRI is one or more of duloxetine (+)-(S)—N-Methyl-3-(naphthalen-1-yloxy)-3-(thiophen-2-yl)propan-1-amine), venlafaxine ((RS)-1-[2-dimethylamino-1-(4-methoxyphenyl)-ethyl]cyclohexanol), nefazodone (1-(3-[4-(3-chlorophenyl)piperazin-1-yl]propyl)-3-ethyl-4-(2-phenoxyethyl)-1H-1,2,4-triazol-5(4H)-one), and milnacipran ((1R*,2S*)-2-(aminomethyl)-N,N-diethyl-1-phenylcyclopropanecarboxamide). These agents are approved by the U.S. FDA for various uses. For example, the present disclosure encompasses doses of duloxetine of doses of above 60 mg daily or above 80 mg daily or above 100 mg daily. Further, SNRIs are also provided in certain embodiments, including, for example, desvenlafaxine, tramadol, and sibutramine.

In some embodiments the In some embodiments, the additional therapeutic agent comprises dopamine active augmenting agents. In some embodiments dopamine active augmenting agents include agents that may be used in the treatment of depression or other mood disorders and which have been shown to boost the antidepressant effect of a main antidepressant treatment. In some embodiments, the dopamine active augmenting agent is one or more of an amphetamine salt, pramipexole, and ropinirole. Amphetamine salts include, for example, ADDERALL, a combination of four amphetamine salts (racemic amphetamine aspartate monohydrate, racemic amphetamine sulfate, dextroamphetamine saccharide, and dextroamphetamine sulfate). ADDERALL, without wishing to be bound by theory, is a dopamine releasing agent, a norepinephrine releasing agent, and can be mildly serotonergic. Pramipexole ((S)-N6-propyl-4,5,6,7-tetrahydro-1,3-benzothiazole-2,6-diamine)), without wishing to be bound by theory, is a dopamine agonist of the non-ergoline class. Ropinirole (4-[2-(dipropylamino)ethyl]-1,3-dihydro-2H-indol-2-one)), without wishing to be bound by theory, is also a dopamine agonist of the non-ergoline class of medications.

In some embodiments, the In some embodiments, the additional therapeutic agent comprises selective serotonin re-uptake inhibitors (SSRIs). SSRIs include agents which act upon, and increase, the levels of the neurotransmitter serotonin, which plays an important role in mood. In some embodiments, the SSRI is one or more of citalopram ((RS)-1-[3-(dimethylamino)propyl]-1-(4-fluorophenyl)-1,3-dihydroisobenzofuran-5-carbonitrile), Dapoxetine (S)—N,N-dimethyl-3-(naphthalen-1-yloxy)-1-phenylpropan-1-amine), S-Citalopram ((S)-1-[3-(dimethylamino)propyl]-1-(4-fluorophenyl)-1,3-dihydroisobenzofuran-5-carbonitrile), Fluoxetine (RS)—N-methyl-3-phenyl-3-[4-(trifluoromethyl)phenoxy]propan-1-amine), Fluvoxamine ((E)-5-methoxy-1-[4-(trifluoromethyl)phenyl]pentan-1-one O-2-aminoethyl oxime), Indalpine (3-(2-piperidin-4-ylethyl)-1H-indole), Paroxetine ((3S,4R)-3-[(2H-1,3-benzodioxol-5-yloxy)methyl]-4-(4-fluorophenyl)piperidine), and Zimelidine ((Z)-3-(4-bromophenyl)-N,N-dimethyl-3-(pyridin-3-yl)prop-2-en-1-amine). Further SSRIs are also provided in certain embodiments, including, for example, citalopram, escitalopram, paroxetine, fluoxetine, and fluvoxamine.

In various embodiments, the additional therapeutic agent comprises serotonin antagonist and reuptake inhibitors (SARIs), such as, for example, etoperidone, lubazodone, nefazodone, and trazodone.

In various embodiments, the additional therapeutic agent comprises norepinephrine reuptake inhibitors (NRIs), such as, for example, atomoxetine, reboxetine, and viloxazine. In still other embodiments, further agents may include norepinephrine-dopamine reuptake inhibitors (NDRIs), such as, for example, bupropion, dexmethylphenidate, methylphenidate, and methylphenidate.

In various embodiments, the additional therapeutic agent comprises norepinephrine-dopamine releasing agents (NDRAs), such as, for example, amphetamine, various amphetamine salts (e.g. salts of racemic amphetamine and dextroamphetamine, Adderall), dextroamphetamine, dextromethamphetamine, lysine-amphetamine (e.g. Vyvanase) and lisdexamfetamine.

In various embodiments, the additional therapeutic agent comprises tricyclic antidepressants (TCAs), such as, for example, amitriptyline, butriptyline, clomipramine, desipramine, dosulepin, doxepin, imipramine, iprindole, lofepramine, melitracen, nortriptyline, opipramol, protriptyline, and trimipramine.

In various embodiments, the additional therapeutic agent comprises tetracyclic antidepressants (TeCAs), such as, for example, amoxapine, maprotiline, mianseri, and mirtazapine.

In various embodiments, the additional therapeutic agent comprises monoamine oxidase inhibitors (MAOIs), such as, for example, isocarboxazid, moclobemide, phenelzine, pirlindole, selegiline, and tranylcypromine.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of a stimulus, relative to the absence of such stimulus.

As used herein, the word “include,” and its variants, is intended to be non-limiting, such that recitation of items in a list is not to the exclusion of other like items that may also be useful in the treatments and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

An effective amount of the treatment as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: High-Frequency and Low-Intensity Patterned Transcranial Magnetic Stimulation Over Left Dorsolateral Prefrontal Cortex as Treatment for Major Depressive Disorder: A Report of 3 Cases

The purpose of this report was to ascertain whether repetitive transcranial magnetic stimulation can improve patients' depressive and anxious symptoms, as well as their cognitive performance, sleep symptoms and their general health perception.

Three patients with previous diagnosis of major depressive disorder were invited to participate in this protocol, once they were recruited, clinical history and physical examination were performed.

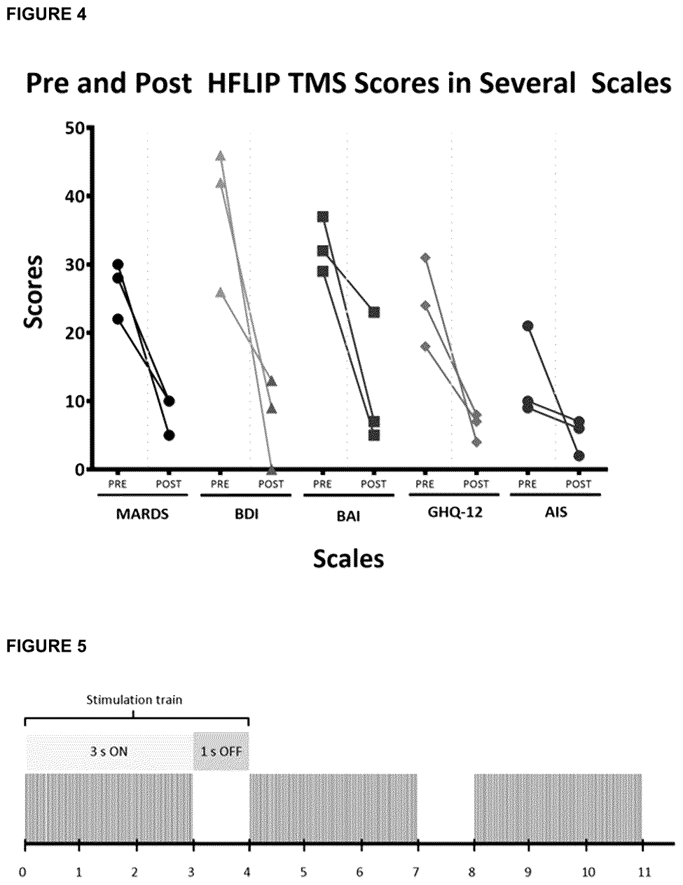

The patients received HFLI TMS stimulation for 45 minutes per treatment, daily, 5-days a week, making a total of 15 sessions distributed in 3 weeks. The device was used by placing a 60 mm circular coil connected to a device (which was connected to an electric current) on the patient's head. The HFLI TMS device emitted a pulsating magnetic field of 550-600 Hz of frequency with an intensity of 0.5 milliteslas (5000 milgauss) during 3 seconds alternate with one second without stimulation, applying a total of 675 trains of stimulation; an example of the stimulation pattern is depicted in FIG. 4.

A baseline assessment was done in all patients before starting HFLI TMS sessions and after the treatment. The following tests were applied during both of the patient's evaluation: Montgomery-Åsberg Depression Rating Scale (MADRS), Beck Depression Inventory (BDI), Beck Anxiety Inventory (BAI), 12 item General Health Questionnaire (GHQ-12), Mini Mental State Evaluation (MMSE), MMSE), and the Athens Insomnia Scale (AIS).

The results of the study in Case 1 showed a significant improvement in all of the measures, including a change from moderate to mild depression in the MADRS and BDI scores, a change from severe to moderate anxiety in the BAI score and a return to normal in the AIS and MMSE scores. Likewise, Case 2 the BDI and MADRS scores were significantly reduced from severe depression to minimal levels, also the BAI score showed the same effect changing from severe to minimal and an absence of insomnia after the application of HFLI TMS. Lastly, in case number 3, scores of the BDI and MADRS were reduced from severe and moderate levels to minimal and mild levels respectively; also, a reduction of severe levels of anxiety to minimal according to the BAI score was observed, as well as a reduction of insomnia levels and an improvement in cognitive functioning according to the MMSE. These results are depicted in FIG. 5, showing the comparison between baseline evaluations and the evaluation after intervention.

Results show that HFLI magnetic fields lead to improvement in depressive and anxious symptoms, including insomnia as well as cognitive performance in patients with Mild Depressive Disorder.

### Example 2: High Frequency Low Intensity (HFLI) Transcranial Magnetic Stimulation Over the Left Prefrontal Dorsolateral Cortex for the Treatment of Treatment-Resistant Major Depressive Disorder Outside a Clinical Setting: A Randomized Controlled Double-Blind Sham Controlled Clinical Trial

The proposed clinical trial is a pivotal multicenter prospective randomized parallel-group triple-blind sham-controlled clinical trial to assess the efficacy of high-frequency low intensity transcranial magnetic stimulation over the left prefrontal dorsolateral cortex for the treatment of treatment-resistant major depressive disorder. The study hypothesis is that 6 weeks of twice-daily HFLI TMS is superior to 6 weeks of twice-daily sessions of sham HFLI TMS in decreasing major depressive symptoms—assessed by Montgomery-Åsberg Depression Rating Scale—compared to baseline.

The primary endpoint of the proposed trial is to assess the short-term safety and efficacy of 6 weeks of twice daily sessions of HFLI TMS over the left prefrontal dorsolateral cortex compared to 6 weeks of twice daily sessions of sham HFLI TMS in diminishing depressive symptoms in subjects suffering from treatment-resistant major depressive disorder outside a clinical setting.

The secondary endpoints of the proposed trial are to assess the long-term safety and efficacy of 6 weeks of twice daily sessions of HFLI TMS over the left prefrontal dorsolateral cortex compared to 6 weeks of twice daily sessions of sham HFLI TMS in diminishing depressive symptoms in subjects suffering from treatment-resistant major depressive disorder outside a clinical setting and to assess the efficacy of HFLI TMS over the left prefrontal dorsolateral cortex compared to sham HFLI TMS in reaching remission of MDD in subjects suffering from treatment-resistant major depressive disorder outside a clinical setting.

The primary efficacy endpoint of the proposed trial is the change from baseline to end of week 6 in Montgomery-Åsberg Depression rating scale (MADRS) score

Secondary efficacy of the proposed trial are change from baseline to the end of week 12 in the Montgomery-Åsberg Depression rating scale (MADRS) score and difference between groups in the proportion of patients that achieved remission. A total score of less than 10 points in the Montgomery-Åsberg Depression rating scale (MADRS) score at the end of week 6 will be considered as remission of the major depressive disorder episode

The intervention of the study are twice-daily sessions 30 minutes sessions of HFLI TMS for 6 weeks (84 sessions). The frequency of the stimulation will be 575 Hz and the intensity of the stimulation will be 5000 miligauss. Depending on the allocated group (real HFLI TMS vs sham HFLI TMS) the devices will emit the magnetic pulses or not.

The results of the proposed trial will provide enough information to show that HFLI TMS used outside a clinical setting is a viable and clinically effective intervention for the treatment of treatment resistant depression.

### Example 3: Open Label Results of the Utilization of High-Frequency and Low-Intensity Patterned Transcranial Magnetic Stimulation Over Left Dorsolateral Prefrontal Cortex as Treatment for Major Depressive Disorder

The purpose of this example is to show how HFLI TMS can be used in a clinical setting to treat treatment resistant depression. In this study, 2 different doctors treated a total of 29 patients suffering from treatment resistant depression. All patients had a stable dose of antidepressant before treatment and received at least 20 sessions of HFLI TMS.

The HFLI TMS device emitted a pulsating magnetic field of 550-600 Hz of frequency with an intensity of 0.5 milliteslas (5000 milgauss) for 3 seconds alternate with one second without stimulation, applying a total of 675 trains of stimulation; an example of the stimulation pattern is depicted in FIG. 4. The site of stimulation for all patients was the prefrontal dorsolateral left cortex.

A baseline assessment of MADRS the was done in all patients before starting HFLI TMS sessions and after the treatment. The baseline average MADRS was 25.44 with a standard deviation of 8.42. After the TMS sessions the average MADRS was 10.27 with a standard deviation of 4.17. This represents almost a 60% improvement in MADRS score.

Results show that HFLI magnetic fields lead to improvement in MADRS score in patients with treatment resistant depression treated in a clinical setting.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

American Psychiatric Association. 2013. Diagnostic and Statistical Manual of Mental Disorders (DSM-5®). American Psychiatric Pub.
GBD 2016 Disease and Injury Incidence and Prevalence Collaborators. 2017. “Global, Regional, and National Incidence, Prevalence, and Years Lived with Disability for 328 Diseases and Injuries for 195 Countries, 1990-2016: A Systematic Analysis for the Global Burden of Disease Study 2016.” The Lancet 390 (10100): 1211-59.
“Major Depression.” n.d. Accessed May 3, 2021. On World Wide Web at nimh.nih.gov/health/statistics/major-depression.
Otte, Christian, Stefan M. Gold, Brenda W. Penninx, Carmine M. Pariante, Amit Etkin, Maurizio Fava, David C. Mohr, and Alan F. Schatzberg. 2016. “Major Depressive Disorder.” Nature Reviews Disease Primers. On World Wide Web at //doi.org/10.1038/nrdp.2016.65.

What is claimed is:

1. A repetitive transcranial magnetic stimulation (rTMS) method for treating or preventing major depressive disorder (MDD) or persistent depressive disorder (PDD), comprising repetitively applying a magnetic pulse to the scalp of a patient in need thereof thereby stimulating neurons in the brain of the patient, wherein the magnetic pulse is applied:
repetitively over the patient's brain; and
at a frequency of about 100 to about 1000 Hz and an intensity of about 1,000 to about 15,000 milligauss.

2. A rTMS method for slowing or preventing a conversion of MDD to PDD, comprising repetitively applying a magnetic pulse to the scalp of a patient in need thereof thereby stimulating neurons in the brain of the patient, wherein the magnetic pulse is applied:
repetitively over the patient's brain; and
at a frequency of about 100 to about 1000 Hz and an intensity of about 1,000 to about 15,000 milligauss.

3. The method of either claim 1 or 2, wherein the magnetic pulse is applied at a frequency of about 400 to about 600 Hz.

4. The method of claim 3, wherein the magnetic pulse is applied at a frequency of about 575 Hz.

5. The method of any one of the previous claims, wherein the magnetic pulse is applied at an intensity of about 10,000 milligauss.

6. The method of any one of the previous claims, wherein the magnetic pulse generates an electric field of about 0.1 to about 10 V/m⁷.

7. The method of claim 6, wherein the magnetic pulse generates an electric field of about 0.5 to about 1.5 V/m⁷.

8. The method of claim 7, wherein the magnetic pulse generates an electric field of about 1 V/m⁷.

9. The method of any one of the previous claims, wherein the method is undertaken once, or twice, or thrice, or four times daily.

10. The method of claim 9, wherein the method is undertaken twice daily.

11. The method of any one of the previous claims, wherein the method is undertaken for greater than about 15 minutes.

12. The method of any one of the previous claims, wherein the method is undertaken for about 15 to about 60 minutes.

13. The method of claim 12, wherein the method is undertaken for about 30 minutes.

14. The method of any one of the previous claims, wherein the pulses are applied about 300 to about 400 times.

15. The method of claim 14, wherein the pulses are applied about 360 times.

16. The method of any one of the previous claims, wherein the pulses are applied discontinuously.

17. The method of any one of the previous claims, wherein the pulses last for about four seconds.

18. The method of claim 17, wherein the pulses last for about four seconds and followed by one second without pulsing.

19. The method of any one of the previous claims, wherein the treatment is applied chronically.

20. The method of any one of the previous claims, wherein the treatment is applied for greater than about 2 months.

21. The method of claim 20, wherein the treatment is applied for greater than about 6 months.

22. The method of claim 21, wherein the treatment is applied for greater than about one year.

23. The method of any one of the previous claims, wherein the treatment is self-applied.

24. The method of any one of the previous claims, wherein the magnetic pulse is applied using a device, the device being suitable for conducting electric current through a coil.

25. The method of claim 24, wherein the device generates a magnetic field.

26. The method of claim 25, wherein the device is suitable for home use.

27. The method of claim 26, wherein the device is portable.

28. The method of any one of the previous claims, wherein the patient is afflicted with MDD.

29. The method of claim 28, wherein the patient is afflicted with PDD.

30. The method of any one of the preceding claims, wherein the patient presents as having the diagnosis of MDD according to DSM V.

31. The method of any one of the preceding claims, wherein the patient presents as having a test score of one or more identified instruments for assessment of MDD or PDD symptoms that is considered to indicate the presence of MDD or PDD, optionally selected from Table 1.

32. The method of any one of the preceding claims, wherein the method provides an improvement in a test score of one or more identified instruments for assessment of MDD or PDD symptoms that is considered to indicate the presence of MDD or PDD, optionally selected from Table 1.

33. The method of any one of the preceding claims, wherein the method stimulates neurons around about 2 to about 3 cm from the skull.

34. The method of any one of the preceding claims, wherein the magnetic pulse is applied repetitively over the patient's left prefrontal dorsolateral cortex.

35. The method of any one of the preceding claims, wherein the method stimulates neurons throughout the patient's brain.

36. The method of any one of the preceding claims, wherein the method substantially stimulates neurons in the left hemisphere of the patient's brain and/or frontal lobe of the patient's brain.

37. The method of any one of the preceding claims, wherein the method substantially stimulates neurons in the cerebral cortex of the patient.

38. The method of any one of the preceding claims, wherein the method stimulates neurons outside of the patient's left prefrontal dorsolateral cortex.

39. The method of any one of the preceding claims, wherein the method prevents or delays the progression of MDD to PDD.

40. The method of any one of the preceding claims, wherein the treatment improves depressive symptoms of the patient.

41. The method of any one of claims 1-40, wherein the treatment prevents diminution of depressive traits of the patient.

42. The method of any one of the preceding claims, wherein the treatment is used in tandem with one or more additional agents.

43. The method of any one of the preceding claims, wherein the treatment obviates the need for treatment with one or more additional agents.

44. The method of any one of the preceding claims, wherein the treatment is substantially free of adverse effects, optionally selected from epileptic seizures, nausea, and headache.
